(19) United States
(12) Patent Application Publication
(10) Pub. No.: US 2014/0276003 A1
(43) Pub. Date: Sep. 18, 2014

(54) Systems and Methods to Detect and Present Interventional Devices via Ultrasound Imaging

(71) Applicant: Chison Medical Imaging Co., Ltd., Wuxi City (CN)

(72) Inventors: Hong Wang, Wuxi City (CN); Ruoli Mo, Wuxi City (CN)

(73) Assignee: Chison Medical Imaging Co., Ltd., Wuxi City (CN)

(21) Appl. No.: 14/209,570

(22) Filed: Mar. 13, 2014

Related U.S. Application Data
(60) Provisional application No. 61/790,586, filed on Mar. 15, 2013.

(51) Int. Cl. A61B 8/08; A61B 8/12; A61B 8/00

(52) U.S. Cl. CPC A61B 8/5253; A61B 8/463; A61B 8/12; A61B 8/4483; A61B 8/0841; A61B 8/5207
USPC 600/424; 600/440

(57) ABSTRACT

The present disclosure includes a method for providing real-time guidance to an interventional device coupled to an ultrasound imaging system operating in a first mode and a second mode. The method includes, in the first mode, stopping transmission of ultrasound signals from a transducer of the ultrasound imaging apparatus, and transmitting, via an acoustic sensor mounted on a head portion of an interventional device, an ultrasound signal that is then received by the transducer to generate a first image of a location of the head portion; in a second mode, stopping transmitting ultrasound signals from the acoustic sensor, transmitting ultrasound signals via the transducer, and receiving echoes of the transmitted ultrasound signals to generate a second image of an object structure; and combining the first image with the second image to derive a third image displaying and highlighting a relative location of the head portion in the object structure.

### CROSS REFERENCE TO RELATED PATENT APPLICATIONS

This application claims the priority and benefit of U.S. Provisional Application No. 61/790,586, filed on Mar. 15, 2013, titled “Systems and Methods to Detect and Present Interventional Devices via Ultrasound Imaging,” which is incorporated in its entirety by reference herein.

### TECHNICAL FIELD

The present disclosure relates to ultrasound imaging in general and, more particularly, to methods and systems for using an acoustic sensor to provide guidance to an interventional device, such as a needle, a catheter, etc., via ultrasound imaging.

### BACKGROUND

Using ultrasound to guide diagnostic or therapeutic invasive procedures involving interventional devices (e.g., needles or catheters) has become increasingly popular in the clinical fields. Interventional ultrasound requires accurately locating the tip or head of an interventional device via ultrasound imaging. Some existing technologies suggest mounting an electrical sensor on the tip of an interventional device to collect an electrical signal from the heart. Those existing technologies, however, have limitations. Often, an interventional device is placed near a target where no or very weak heart signal can be collected, and thus the accurate location of the tip of the interventional device cannot be detected and presented in an ultrasound image. Other existing technologies suggest mounting an electrical sensor on the tip of an interventional device to receive an ultrasonic pulse transmitted from an imaging transducer, convert the pulse into an electrical signal, and pass the signal back to the ultrasound device. Under those existing technologies, however, visualizing the tip of an interventional device in an ultrasound image is difficult when strong tissue clutters are present in the image to weaken the ultrasonic pulse. Also, in those existing technologies, it is difficult to accurately determine which transmitted acoustic beam triggers the electrical sensor, and thus the accurate location of the tip of the interventional device cannot be detected. Moreover, because the ultrasonic pulse traveling in a human or animal body is attenuated very fast and becomes weak and not stable, it is difficult for those existing technologies to distinguish a noise from a real pulse signal at the tip of the interventional device. In sum, the existing technologies can only calculate an approximate, not accurate, location of the tip of the interventional device.

Thus, there is a need to develop a method and system for easily and accurately detecting and presenting the position of interventional devices, such as needles, catheters, etc., via ultrasound imaging and overcome the limitations of prior art systems.

### SUMMARY

The present disclosure includes an exemplary method for providing real-time guidance to an interventional device coupled to an ultrasound imaging system operating in a first mode and a second mode. Embodiments of the method include, in the first mode: stopping transmission of ultrasound signals from a transducer of the ultrasound imaging system; transmitting, via an acoustic sensor mounted on a head portion of the interventional device, an ultrasound signal; receiving, via the transducer, the transmitted ultrasound signal; and generating a first image of a location of the head portion based on the received ultrasound signal. Embodiments of the method also include, in the second mode: stopping transmitting ultrasound signals from the acoustic sensor; transmitting, via the transducer, ultrasound signals; receiving echoes of the transmitted ultrasound signals reflected back from an object structure; and generating a second image of the object structure based on the received echoes. Embodiments of the method further include combining the first image with the second image to derive a third image displaying a location of the head portion relative to the object structure. Some embodiments of the method also include highlighting the relative location of the head portion in the third image by brightening the location, coloring the location, or marking the location using a text or sign.

An exemplary system in accordance with the present disclosure comprises a transducer, a processor coupled to the transducer, and an acoustic sensor mounted on a head portion of an interventional device. When the disclosed system operates in a first mode, the transducer stops transmitting ultrasound signals, and the acoustic sensor transmits an ultrasound signal that is then received by the transducer and is used to generate a first image of a location of the head portion. When the disclosed system operates in a second mode, the acoustic sensor stops transmitting ultrasound signals, and the transducer transmits ultrasound signals and receives echoes of the transmitted ultrasound signals that are used to generate a second image of an object structure. In some embodiments, the processor combines the first image with the second image to derive a third image displaying a location of the head portion relative to the object structure. In certain embodiments, the processor highlights the relative location of the head portion in the third image by brightening the location, coloring the location, or marking the location using a text or sign.

It is to be understood that both the foregoing general description and the following detailed description are exemplary and explanatory only and are not restrictive of the invention, as claimed.

### BRIEF DESCRIPTION OF THE DRAWINGS

FIG. 1 illustrates a block diagram of an exemplary system consistent with the present disclosure.

FIG. 2 is a block diagram illustrating an embodiment of the exemplary system of FIG. 1.

FIG. 3 is a functional diagram illustrating an exemplary process flow in the embodiment of FIG. 2.

FIG. 4 is a functional diagram illustrating another exemplary process flow in the embodiment of FIG. 2.

FIG. 5 illustrates an exemplary sensor image.

FIG. 6 illustrates an exemplary ultrasound image.

FIG. 7 illustrates an exemplary enhanced visualization image combining the sensor image of FIG. 5 with the ultrasound image of FIG. 6.

FIG. 8 illustrates a series of exemplary enhanced visualization images generated in real-time.

FIG. 9 is a flowchart representing an exemplary method of using an acoustic sensor to provide guidance to an interventional device via ultrasound imaging.

### DETAILED DESCRIPTION

Reference will now be made in detail to the exemplary embodiments illustrated in the accompanying drawings. Wherever possible, the same reference numbers will be used throughout the drawings to refer to the same or like parts.

Methods and systems disclosed herein address the above described needs. For example, exemplary embodiments include an acoustic sensor mounted on a head portion of an interventional device, such as a needle, a catheter, etc. The acoustic sensor is used as a beacon. Instead of receiving an electrical signal from the heart or receiving an acoustic pulse from an imaging transducer, the acoustic sensor disclosed herein will be a part of an ultrasound imaging system to transmit acoustic pulses. In a first mode of the ultrasound imaging system, the imaging transducer itself does not transmit acoustic pulses or transmits with zero power. Instead, the system instructs the acoustic sensor to transmit acoustic pulses with the timing as if it were located at the center of the transmitting aperture of the imaging transducer to form a sensor image. The transmitting aperture comprises one or more transducer elements. The sensor image, which is a two-dimensional (“2D”) or three-dimensional (“3D”) image, is formed as if the transducer is transmitting. As a result, a one-way point spread function (“PSF”) of the acoustic sensor can be seen on the sensor image. The imaging depth should be multiplied by two due to the one-way characteristics. This sensor image can be combined with an ultrasound image of an object structure to derive an enhanced visualization image, which shows a location of the head portion of the interventional device relative to the object structure. The acoustic pulses transmitted by the acoustic sensor disclosed herein are much stronger and more stable than an acoustic beam transmitted by a transducer element and an echo of the beam, and can be easily and accurately detected and recorded in the sensor image. Methods and systems disclosed herein provide a real-time and accurate position of a head portion of an interventional device in live ultrasound imaging.

FIG. 1 illustrates a block diagram of an exemplary system 100 consistent with the present disclosure. Exemplary system 100 can be any type of system that provides real-time guidance to an interventional device via ultrasound imaging in a diagnostic or therapeutic invasive procedure. Exemplary system 100 can include, among other things, an ultrasound apparatus 100A having an ultrasound imaging field 120, and an acoustic sensor 112 mounted on a head portion of an interventional device 110 coupled to ultrasound apparatus 100A. Acoustic sensor 112 can be coupled to ultrasound apparatus 100A directly or through interventional device 110.

Ultrasound apparatus 100A can be any device that utilizes ultrasound to detect and measure an object located within the scope of ultrasound imaging field 120, and presents the measured object in an ultrasonic image. The ultrasonic image can be in gray-scale, color, or a combination thereof, and can be 2D or 3D.

Interventional device 110 can be any device that is used in a diagnostic or therapeutic invasive procedure. For example, interventional device 110 can be provided as a needle, a catheter, or any other diagnostic or therapeutic device.

Acoustic sensor 112 can be any device that transmits acoustic pulses or signals (i.e., ultrasound pulses or signals), which are converted from electrical pulses. For example, acoustic sensor 112 can be a type of microelectromechanical systems (“MEMS”). In some embodiments, acoustic sensor 112 can also receive acoustic pulses transmitted from another device.

FIG. 2 is a block diagram illustrating ultrasound apparatus 100A in greater detail within exemplary system 100. Ultrasound apparatus 100A includes a display 102, ultrasound transducer 104, processor 106, and ultrasound beamformer 108. The illustrated configuration of ultrasound apparatus 100A is exemplary only, and persons of ordinary skill in the art will appreciate that the various illustrated elements may be provided as discrete elements or be combined, and be provided as any combination of hardware and software.

With reference to FIG. 2, ultrasound transducer 104 can be any device that has multiple piezoelectric elements to convert electrical pulses into an acoustic beam to be transmitted and to receive echoes of the transmitted acoustic beam. The transmitted acoustic beam propagates into a subject (such as a human or animal body), where echoes from interfaces between object structures (such as tissues within a human or animal body) with different acoustic impedances are reflected back to the transducer. Transducer elements convert the echoes into electrical signals. Based on the time differences between the acoustic beam transmission time and the echo receiving time, an image of the object structures can be generated.

Ultrasound beamformer 108 can be any device that enables directional or spatial selectivity of acoustic signal transmission or reception. In particular, ultrasound beamformer 108 focuses acoustic beams to be transmitted to point in a same direction, and focuses echo signals received as reflections from different object structures. In some embodiments, ultrasound beamformer 108 delays the echo signals arriving at different elements and aligns the echo signals to form an isophase plane. Ultrasound beamformer 108 then sums the delayed echo signals coherently. In certain embodiments, ultrasound beamformer 108 may perform beamforming on electrical or digital signals that are converted from echo signals.

Processor 106 can be any device that controls and coordinates the operation of other parts of ultrasound apparatus 100A, processes data or signals, generates ultrasound images, and outputs the generated ultrasound images to a display. In some embodiments, processor 106 may output the generated ultrasound images to a printer, or remote device through a data network. For example, processor 106 can be a central processing unit (CPU), a microprocessor, an application specific integrated circuit (ASIC), a field-programmable gate array (FPGA), a complex programmable logic device (CPLD), a printed circuit board (PCB), a digital signal processor (DSP), etc.

Display 102 can be any device that displays ultrasound images. For example, display 102 can be a monitor, display panel, projector, or any other display device. In certain embodiments, display 102 can be a touchscreen display with which a user can interact through touches. In some embodiments, display 102 can be a display device with which a user can interact by remote gestures.

FIG. 3 is a functional diagram illustrating an exemplary process flow for generating a sensor image in exemplary system 100, which operates in a first mode. In the first mode, system 100 performs one frame or volume imaging with zero transmit power to ultrasound transducer 104. However, the system sends a transmit signal to acoustic sensor 112, which can be treated as an element of the transducer to transmit ultrasound signals. This frame or volume is for acoustic sensor visualization. Thus, in the first mode, ultrasound transducer 104 does not transmit ultrasound signals, but acoustic sensor 112 transmits ultrasound signals and ultrasound transducer 104 receives them. It will now be appreciated by one of ordinary skill in the art that the illustrated process flow can be altered to modify steps, delete steps, or include additional steps.

After receiving electrical pulses provided (302) by ultrasound apparatus 100A, acoustic sensor 112 transmits (304) to ultrasound transducer 104 acoustic pulses (ultrasound signals) that are converted from the electrical pulses. The conversion can be performed by acoustic sensor 112 or another component. Upon receiving (304) the acoustic pulses transmitted from acoustic sensor 112, ultrasound transducer 104 converts the received acoustic pulses into electrical signals, which are forwarded (306) to ultrasound beamformer 108. In some embodiments, the electrical signals are converted into digital signals and are then forwarded (306) to ultrasound beamformer 108 for beamforming.

Following a beamforming process, ultrasound beamformer 108 transmits (308) the processed electrical or digital signals to processor 106, which processes the signals to generate an image of a one-way point spread function (“PSF”) of acoustic sensor 112. FIG. 5 illustrates an exemplary sensor image 500 that processor 106 generates. As shown in FIG. 5, a bright spot 502 indicates an image of a one-way PSF of acoustic sensor 112, which is also a location of the head portion of interventional device 110, on which acoustic sensor 112 is mounted.

Referring back to FIG. 3, unlike regular ultrasound imaging in which an acoustic signal travels a round trip between a transducer and an object, in forming the sensor image, the acoustic pulses travel one way from acoustic sensor 112 to ultrasound transducer 104. Thus, in generating the sensor image, a depth (which indicates a distance between transducer 104 and acoustic sensor 112) or a velocity of the acoustic pulses should be doubled.

In some embodiments, the sensor image can include a unique identifier (image ID) for later retrieval and association purpose. In some embodiments, the sensor image can be stored in a storage or database for later processing.

FIG. 4 is a functional diagram illustrating an exemplary process flow for generating an ultrasound image in exemplary system 100, which now operates in a second mode. In the second mode, acoustic sensor 112 does not transmit ultrasound signals, but ultrasound transducer 104 transmits ultrasound signals and receives their echoes. It will now be appreciated by one of ordinary skill in the art that the illustrated process flow can be altered to modify steps, delete steps, or include additional steps.

Under beamforming control (402) of ultrasound beamformer 108, ultrasound transducer 104 transmits (404) ultrasound signals and receives (406) echo signals reflected from an object structure (e.g., a tissue, organ, bone, muscle, tumor, etc. of a human or animal body) in ultrasound imaging field 120. Ultrasound transducer 104 converts the received echo signals into electrical signals, which are passed (408) to ultrasound beamformer 108. In some embodiments, the electrical signals are converted into digital signals and are then passed (408) to ultrasound beamformer 108 for beamforming.

Following a beamforming process, ultrasound beamformer 108 transmits (410) the processed electrical or digital signals to processor 106, which processes the signals to generate an ultrasound image of the object structure. FIG. 6 illustrates an exemplary ultrasound image 600 of an object structure. As shown in FIG. 6, an object structure 602 is visualized in ultrasound image 600.

Referring back to FIG. 3, in some embodiments, the ultrasound image of the object structure can include a unique identifier (image ID) for later retrieval and association purpose. In some embodiments, the ultrasound image can be stored in a storage or database for later processing.

Processor 106 combines the sensor image generated in the first mode with the ultrasound image generated in the second mode to derive an enhanced visualization image, which is outputted (412) to display 102. In some embodiments, processor 106 retrieves the sensor image stored in a storage or database based on an image ID, which corresponds to an image ID of the ultrasound image, to derive the enhanced visualization image. In certain embodiments, the enhanced visualization image can include a unique identifier (image ID) for later retrieval and association purpose. In some embodiments, the enhanced visualization image can be stored in a storage or database for later processing.

Since the sensor image has a same size as the ultrasound image, in some embodiments, processor 106 derives the enhanced visualization image based on a sum of pixel values in corresponding coordinates of the sensor image and the ultrasound image. For example, processor 106 can perform a pixel-by-pixel summation. That is, processor 106 adds a pixel value at a coordinate of the sensor image to a pixel value at a corresponding coordinate of the ultrasound image to derive a pixel value for the enhanced visualization image, and then computes a next pixel value for the enhanced visualization image in a similar manner, and so on.

In other embodiments, processor 106 derives the enhanced visualization image based on a weighted pixel-by-pixel summation of pixel values at corresponding coordinates of the sensor image and the ultrasound image. For example, processor 106 applies a weight value to a pixel value of the sensor image and applies another weight value to a corresponding pixel value of the ultrasound image, before performing the pixel summation.

In certain embodiments, processor 106 derives the enhanced visualization image based on computing maximum values of corresponding pixels of the sensor image and the ultrasound image. For example, processor 106 determines a maximum value by comparing a pixel value at a coordinate of the sensor image to a pixel value at a corresponding coordinate of the ultrasound image, and uses the maximum value as a pixel value for the enhanced visualization image. Processor 106 then computes a next pixel value for the enhanced visualization image in a similar manner, and so on.

With reference to FIG. 4, the enhanced visualization image shows a location of acoustic sensor 112 (i.e., a location of a head portion of interventional device 110) relative to the object structure. In some embodiments, the enhanced visualization image highlights the location by, for example, brightening the location, coloring the location, or marking the location using a text or sign.

FIG. 7 illustrates an exemplary enhanced visualization image 700 combining sensor image 500 of FIG. 5 with ultrasound image 600 of FIG. 6. As shown in FIG. 7, enhanced visualization image 700 shows and highlights a location of the head portion of interventional device 110 relative to object structure 602.

FIG. 8 illustrates a series of exemplary enhanced visualization images 700 that are generated to provide real-time guidance to interventional device 110 via ultrasound imaging. As shown in FIG. 8, at each point of time, ultrasound apparatus 100A combines an ultrasound image 600 with a previously generated sensor image 500 to derive an enhanced visualization image 700, and combines the ultrasound image 600 with a next generated sensor image 500 (if any) to derive a next enhanced visualization image 700. In some embodiments, ultrasound apparatus 100A retrieves and associates a sensor image 500 with an ultrasound image 600 based on image IDs. For example, ultrasound apparatus 100A retrieves an ultrasound image 600 with an image ID “N” and a sensor image 500 with an image ID “N−1” to derive an enhanced visualization image 700 with an image ID “M.” Similarly, ultrasound apparatus 100A combines the ultrasound image 600 with an image ID “N” with a sensor image 500 with an image ID “N+1” to derive an enhanced visualization image 700 with an image ID “M+1,” and so on. In this way, real-time guidance to interventional device 110 can be provided via live ultrasound imaging. In other embodiments, other methods may be used to retrieve generated sensor images and ultrasound images to derive enhanced visualization images.

FIG. 9 is a flowchart representing an exemplary method of using an acoustic sensor to provide guidance to an interventional device via ultrasound imaging.

It will now be appreciated by one of ordinary skill in the art that the illustrated procedure can be altered to delete steps, change the order of steps, or include additional steps.

After an initial start step, an ultrasound apparatus operates in a first mode, and stops (902) transmission of ultrasound signals from its transducer. In the first mode, the ultrasound apparatus instructs an acoustic sensor mounted on a head portion of an interventional device to transmit (904) an ultrasound signal, and instructs the transducer to receive (906) the ultrasound signal. The ultrasound apparatus generates a first image of the acoustic sensor, indicating a location of the head portion.

In a second mode, the ultrasound apparatus stops (908) transmission of ultrasound signals from the acoustic sensor, and instructs the transducer to transmit ultrasound signals and receive (910) echo signals reflected back from an object structure. Based on the received echo signals, the ultrasound apparatus generates a second image, which is an ultrasound image of the object structure.

The ultrasound apparatus then combines (912) the first image with the second image to derivate a third image, which displays a location of the head portion of the interventional device relative to the object structure. The ultrasound apparatus performs the combination, as explained above.

The ultrasound apparatus displays (914) the third image that may highlight the location of the head portion of the interventional device in the object structure. The process then proceeds to end.

The methods disclosed herein may be implemented as a computer program product, i.e., a computer program tangibly embodied in a non-transitory information carrier, e.g., in a machine-readable storage device, or a tangible non-transitory computer-readable medium, for execution by, or to control the operation of, data processing apparatus, e.g., a programmable processor, a computer, or multiple computers. A computer program may be written in any form of programming language, including compiled or interpreted languages, and it may be deployed in any form, including as a standalone program or as a module, component, subroutine, or other unit suitable for use in a computing environment. A computer program may be deployed to be executed on one computer or on multiple computers at one site or distributed across multiple sites and interconnected by a communication network.

A portion or all of the methods disclosed herein may also be implemented by an application-specific integrated circuit (ASIC), a field-programmable gate array (FPGA), a complex programmable logic device (CPLD), a printed circuit board (PCB), a digital signal processor (DSP), a combination of programmable logic components and programmable interconnects, a single central processing unit (CPU) chip, a CPU chip combined on a motherboard, a general purpose computer, or any other combination of devices or modules capable of performing depth map generation for 2D-to-3D image conversion based on image content disclosed herein.

In the preceding specification, the invention has been described with reference to specific exemplary embodiments. It will, however, be evident that various modifications and changes may be made without departing from the broader spirit and scope of the invention as set forth in the claims that follow. The specification and drawings are accordingly to be regarded as illustrative rather than restrictive. Other embodiments of the invention may be apparent to those skilled in the art from consideration of the specification and practice of the invention disclosed herein.

What is claimed is:

1. An ultrasound imaging system operating in a first mode and a second mode, comprising:
a transducer;
a processor coupled to the transducer; and
an acoustic sensor mounted on a head portion of an interventional device;
wherein in the first mode, the transducer stops transmitting ultrasound signals, and the acoustic sensor transmits an ultrasound signal that is then received by the transducer and is used to generate a first image of a location of the head portion;
wherein in the second mode, the acoustic sensor stops transmitting ultrasound signals, and the transducer transmits ultrasound signals and receives echoes of the transmitted ultrasound signals that are used to generate a second image of an object structure; and
wherein the processor combines the first image with the second image to derive a third image displaying a location of the head portion relative to the object structure.

2. The ultrasound imaging system of claim 1, wherein the interventional device is a needle, a catheter, or any other device used in a diagnostic or therapeutic invasive procedure.

3. The ultrasound imaging system of claim 1, wherein the processor generates the first image showing a one-way point spread function of the acoustic sensor.

4. The ultrasound imaging system of claim 1, wherein the processor derives the third image based on performing a pixel-by-pixel summation of values of corresponding pixels in the first image and the second image to generate pixels of the third image.

5. The ultrasound imaging system of claim 1, wherein the processor derives the third image based on:
applying a first weight value to values of pixels of the first image to acquire weighted pixel values of the first image;
applying a second weight value to values of corresponding pixels of the second image to acquire corresponding weighted pixel values of the second image; and
performing a pixel-by-pixel summation of the weighted pixel values of the first image and the corresponding weighted pixel values of the second image to generate pixels of the third image.

6. The ultrasound imaging system of claim 1, further comprising:
an image database to store the first image in association with the second image, wherein the first image is associated with the second image by a first unique identifier that uniquely identifies the first image,
wherein a second unique identifier is obtained based on the first unique identifier to uniquely identify the associated second image.

7. The ultrasound imaging system of claim 1, wherein the processor highlights the relative location of the head portion in the third image by brightening the location, coloring the location, or marking the location using a text or sign.

8. A computer-implemented method for providing real-time guidance to an interventional device coupled to an ultrasound imaging system operating in a first mode and a second mode, the method comprising:
in the first mode:
stopping transmission of ultrasound signals from a transducer of the ultrasound imaging system,
transmitting, via an acoustic sensor mounted on a head portion of the interventional device, an ultrasound signal,
receiving, via the transducer, the transmitted ultrasound signal, and
generating a first image of a location of the head portion based on the received ultrasound signal;
in the second mode:
stopping transmitting ultrasound signals from the acoustic sensor,
transmitting, via the transducer, ultrasound signals,
receiving echoes of the transmitted ultrasound signals reflected back from an object structure, and
generating a second image of the object structure based on the received echoes; and
combining the first image with the second image to derive a third image displaying a location of the head portion relative to the object structure.

9. The method of claim 8, wherein generating the first image comprises showing a one-way point spread function of the acoustic sensor.

10. The method of claim 8, wherein combining the first image with the second image to derive the third image comprises:
performing a pixel-by-pixel summation of values of corresponding pixels in the first image and the second image to generate pixels of the third image.

11. The method of claim 8, wherein combining the first image with the second image to derive the third image comprises:
applying a first weight value to values of pixels of the first image to acquire weighted pixel values of the first image;
applying a second weight value to values of corresponding pixels of the second image to acquire corresponding weighted pixel values of the second image; and
performing a pixel-by-pixel summation of the weighted pixel values of the first image and the corresponding weighted pixel values of the second image to generate pixels of the third image.

12. The method of claim 8, further comprising:
storing the first image in association with the second image, wherein the first image is associated with the second image by a first unique identifier that uniquely identifies the first image,
wherein a second unique identifier is obtained based on the first unique identifier to uniquely identify the associated second image.

13. The method of claim 12, further comprising:
providing from storage the first image and the associated second image based on the first unique identifier and the second unique identifier for deriving the third image.

14. The method of claim 8, further comprising:
highlighting the relative location of the head portion in the third image by brightening the location, coloring the location, or marking the location using a text or sign.

15. An ultrasound imaging apparatus coupled to an interventional device, comprising:
a transducer to:
in a first mode, stop transmitting ultrasound signals, and receive an ultrasound signal transmitted by an acoustic sensor mounted on a head portion of the interventional device, wherein the received ultrasound signal is used to generate a first image of a location of the head portion, and
in a second mode, transmit ultrasound signals, and receive echoes of the transmitted ultrasound signals reflected back from an object structure, wherein the received echoes are used to generate a second image of the object structure; and
a processor coupled to the transducer to combine the first image with the second image to derive a third image displaying a location of the head portion relative to the object structure.

16. The ultrasound imaging apparatus of claim 15, wherein the processor generates the first image showing a one-way point spread function of the acoustic sensor.

17. The ultrasound imaging apparatus of claim 15, wherein the processor derives the third image based on performing a pixel-by-pixel summation of values of corresponding pixels in the first image and the second image to generate pixels of the third image.

18. The ultrasound imaging apparatus of claim 15, wherein the processor derives the third image based on:
applying a first weight value to values of pixels of the first image to acquire weighted pixel values of the first image;
applying a second weight value to values of corresponding pixels of the second image to acquire corresponding weighted pixel values of the second image; and
performing a pixel-by-pixel summation of the weighted pixel values of the first image and corresponding weighted pixel values of the second image to generate pixels of the third image.

19. The ultrasound imaging apparatus of claim 15, further comprising:
an image database to store the first image in association with the second image, wherein the first image is associated with the second image by a first unique identifier that uniquely identifies the first image,
wherein a second unique identifier is obtained based on the first unique identifier to uniquely identify the associated second image.

20. The ultrasound imaging apparatus of claim 15, wherein the processor highlights the relative location of the head portion in the third image by brightening the location, coloring the location, or marking the location using a text or sign.
